(19) United States
(12) Patent Application Publication
(10) Pub. No.: US 2026/0137321 A1
(43) Pub. Date: May 21, 2026

(54) BIOELECTRODE COMPLEX FOR MEASURING PHYSIOLOGICAL SIGNALS, MANUFACTURING METHOD THEREOF AND WEARABLE DEVICE COMPRISING THE SAME

(71) Applicants: UIF (University Industry Foundation), Yonsei University, Seoul (KR); Daegu Gyeongbuk Institute of Science and Technology, Daegu (KR)

(72) Inventors: Jeong Ho CHO, Seoul (KR); Sang Hyun LEE, Seoul (KR); Dong Hae HO, Seoul (KR); Kyung In JANG, Daegu (KR); Janghwan JEKAL, Daegu (KR)

(73) Assignees: UIF (University Industry Foundation), Yonsei University, Seoul (KR); Daegu Gyeongbuk Institute of Science and Technology, Daegu (KR)

(21) Appl. No.: 19/228,414

(22) Filed: Jun. 4, 2025

(30) Foreign Application Priority Data
Nov. 21, 2024 (KR) 10-2024-0167357

(51) Int. Cl. A61B 5/27; A61B 5/256

(52) U.S. Cl. CPC A61B 5/27; A61B 5/256

(57) ABSTRACT

The present disclosure relates to a bioelectrode complex for measuring physiological signals, a method for manufacturing the same, and a wearable device including the same. The bioelectrode complex for measuring physiological signals of the present disclosure, wherein a cellulose nanofiber and a polycarboxylate-based polymer are inserted between the layers of a MXene nanosheet having a layered structure, is advantageous in that mechanical strength and oxidation stability are remarkably superior and it can be applied to a wearable device due to improved flexibility.

### CROSS-REFERENCE TO RELATED APPLICATION

This application claims priority to Korean Patent Application No. 10-2024-0167357, filed on Nov. 21, 2024, and all the benefits accruing therefrom under 35 U.S.C. § 119, the contents of which in its entirety are herein incorporated by reference.

### BACKGROUND

### 1. Field

The present disclosure relates to a bioelectrode complex for measuring physiological signals, a method for manufacturing the same and a wearable device including the same.

### 2. Description of the Related Art

The importance of wearable technology is growing more and more in the field of healthcare because of social problems such as population aging and the advent of infectious diseases. Preventive medicine has the potential of greatly reducing social cost through early intervention in acute diseases. However, there are several challenges to effective realization of preventive medicine, such as downsizing of physiological data acquisition systems, stable contact between the skin and the bioelectrode, persistent wearability, easiness of data analysis, etc. In order to solve these problems, a wearable healthcare system is emerging as a promising candidate that can effectively acquire and monitor the physiological data of a user.

Among the wearable healthcare systems, tattoo- or fiber-based wearable devices are being researched actively due to the potential for everyday analysis of psychological diseases such as cardiovascular diseases, muscle disorder, Parkinson's disease, Alzheimer's disease, etc. Recently, an elastic and flexible dry electrode, i.e., an electrode consisting of a one-dimensional (1D) or two-dimensional (2D) conductive material, was used in a bodysuit to improve signal sensitivity significantly. However, despite this advantage, there was the problem of oxidation due to exposure to sweat, moisture, etc. when the wearable device was used for a clothing.

Meanwhile, MXenes, which are two-dimensional layered materials with large specific surface area and high electrical conductivity, have the problem that mechanical strength is weak and electrical conductivity is decreased rapidly upon oxidation.

Accordingly, in order to use the two-dimensional layered material MXene as an electrode material, research and development are required to resolve the problem of weak mechanical strength and decrease of electrical conductivity due to oxidation and the problem of oxidation due to sweat, moisture, etc. when applied to a clothing.

### REFERENCES OF THE RELATED ART

### Patent Documents

(Patent document 1) Korean Patent Publication No. 2023-0042625.

### SUMMARY

The present disclosure is directed to providing a bioelectrode complex for measuring physiological signals, with improved mechanical strength and oxidation stability.

In addition, the present disclosure is directed to providing a wearable device including the bioelectrode complex for measuring physiological signals of the present disclosure.

In addition, the present disclosure is directed to providing a clothing for measuring physiological signals, which includes the wearable device of the present disclosure.

In addition, the present disclosure is directed to providing a method for manufacturing a bioelectrode complex for measuring physiological signals.

The present disclosure provides a bioelectrode complex for measuring physiological signals, which includes: a MXene nanosheet having a layered structure; and a cellulose nanofiber and a polycarboxylate-based polymer located between the MXene nanosheet and bound on the surface of the MXene nanosheet.

In addition, the present disclosure provides a wearable device including the bioelectrode complex for measuring physiological signals of the present disclosure.

In addition, the present disclosure provides a clothing for measuring physiological signals, which includes the wearable device of the present disclosure.

In addition, the present disclosure provides a method for manufacturing a bioelectrode complex for measuring physiological signals, which includes: a step of preparing a MXene dispersion containing a MXene nanosheet; a step of preparing a mixture containing the MXene dispersion, a cellulose nanofiber and a polycarboxylate-based polymer; and a step of preparing a bioelectrode complex by coating the mixture on a substrate.

The bioelectrode complex for measuring physiological signals of the present disclosure, wherein a cellulose nanofiber and a polycarboxylate-based polymer are inserted between the layers of a MXene nanosheet having a layered structure, is advantageous in that mechanical strength and oxidation stability are remarkably superior and it can be applied to a wearable device due to improved flexibility.

The effects of the present disclosure are not limited to the effects mentioned above, and it should be understood that all the effects that can be inferred from the following description are encompassed therein.

### BRIEF DESCRIPTION OF THE DRAWINGS

FIG. 1 shows the chemical structure of a bioelectrode complex for measuring physiological signals according to the present disclosure.

FIG. 2 shows the SEM and EDX elemental mapping results of an MX-CNF-PCE electrode complex prepared in Example 1 according to the present disclosure.

FIG. 3 shows the SEM image of a pristine cotton fabric.

FIG. 4A shows the EDX mapping result of pristine MXene electrodes at 50× magnification.

FIG. 4B shows the EDX mapping result of MX-CNF-PCE electrode complexes at 50× magnification.

FIG. 4C shows the EDX mapping result of cotton fabrics at 50× magnification.

FIG. 4D shows the EDX mapping result of cotton fabrics at 5000× magnification.

FIG. 4E shows the EDX mapping result of MX-CNF-PCE electrode complexes at 5000× magnification.

FIG. 5A shows the result of comparing the sheet resistance of MXene-based electrodes, i.e., a MXene electrode, an MX-CNF electrode and an MX-CNF-PCE electrode complex. The bar chart shows sample means (n=8) with standard deviation error bars.

FIG. 5B shows the result of comparing skin impedance between reference dry and wet Ag/AgCl electrodes and pneumatically fully activated FLEXER for a MXene electrode, an MX-CNF electrode and an MX-CNF-PCE electrode complex.

FIG. 6A shows the tensile strain of a MXene electrode, an MX-CNF electrode and an MX-CNF-PCE electrode complex. Sample means (n=5) are shown with standard deviation error bars.

FIG. 6B shows the result of 1000 cycles of 15% bending strain test for a MXene electrode, an MX-CNF electrode and an MX-CNF-PCE electrode complex.

FIG. 7A shows the result of evaluating the oxidation stability of a MXene electrode, an MX-CNF electrode and an MX-CNF-PCE electrode complex under 100% humidity environment. Sample means (n=5) are shown with standard deviation error bars.

FIG. 7B shows the resistance stability of a MXene electrode, an MX-CNF electrode and an MX-CNF-PCE electrode complex to artificial sweat droplets. Sample means (n=5) are shown with standard deviation error bars.

FIG. 8A shows the tensile strain of an MXene electrode and MX-CNF-PCE electrode complexes prepared in Example 1 and Comparative Examples 1 to 3 depending on the mixing ratio of CNF and PCE.

FIG. 8B shows the result of cyclic bending for a MXene electrode and MX-CNF-PCE electrode complexes prepared in Example 1 and Comparative Examples 1 to 3.

FIG. 8C shows the oxidation stability of a MXene electrode and MX-CNF-PCE electrode complexes prepared in Example 1 and Comparative Examples 1 to 3 under 100% humidity environment.

FIG. 8D shows the resistance stability of a MXene electrode and MX-CNF-PCE electrode complexes prepared in Example 1 and Comparative Examples 1 to 3 against artificial sweat droplets.

FIG. 9A shows the XRD spectra of a MXene electrode, an MX-CNF electrode and an MX-CNF-PCE electrode complex.

FIG. 9B shows the XPS spectra of a MXene electrode, an MX-CNF electrode and an MX-CNF-PCE electrode complex.

FIG. 9C compares the XPS analysis result of Ti²⁺/Ti³⁺ area ratio and hydrogen bond ratio for a MXene electrode, an MX-CNF electrode and an MX-CNF-PCE electrode complex referred from the area of XPS deconvoluted peaks.

FIG. 10 shows the extended XRD spectra of a MXene electrode, an MX-CNF electrode and an MX-CNF-PCE electrode complex.

### DETAILED DESCRIPTION

Hereinafter, the exemplary embodiments of the present disclosure will be described more specifically.

The present disclosure relates to a bioelectrode complex for measuring physiological signals, a method for manufacturing the same and a wearable device including the same.

As described above, although the application of an electrode consisting of a one-dimensional (1D) or two-dimensional (2D) conductive material to a bodysuit improves signal sensitivity significantly, there is the problem of oxidation due to exposure to sweat, moisture, etc. when the wearable device is used for a clothing. Meanwhile, MXenes, which are two-dimensional layered materials with large specific surface area and high electrical conductivity, have the problem that mechanical strength is weak and electrical conductivity is decreased rapidly upon oxidation.

The inventors of the present disclosure have found out that a bioelectrode complex for measuring physiological signals prepared by inserting a cellulose nanofiber and a polycarboxylate-based polymer between the layers of a MXene nanosheet having a layered structure is advantageous in that mechanical strength and oxidation stability are remarkably superior and it can be applied to a wearable device due to improved flexibility.

Specifically, the present disclosure provides a bioelectrode complex for measuring physiological signals, which includes: a MXene nanosheet having a layered structure; and a cellulose nanofiber and a polycarboxylate-based polymer located between the MXene nanosheet and bound on the surface of the MXene nanosheet.

FIG. 1 shows the chemical structure of the bioelectrode complex for measuring physiological signals according to the present disclosure. Referring to FIG. 1, in the bioelectrode complex, the cellulose nanofiber is located between the MXene nanosheet having a layered structure. The cellulose nanofiber is hydrogen-bonded to the terminal functional groups of the MXene nanosheet, such as hydroxyl groups, oxygen groups, fluorine groups, etc., etc., so as to contact the opposing surfaces of the MXene nanosheet. And, the polycarboxylate-based polymer is covalently bonded to transition metals present on the opposing surfaces of the MXene nanosheet.

The MXene nanosheet may be represented by Chemical Formula 1.

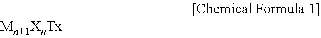

In Chemical Formula 1, M is one or more transition metal selected from a group consisting of titanium (Ti), zirconium (Zr), hafnium (Hf), vanadium (V), chromium (Cr), manganese (Mn), scandium (Sc), molybdenum (Mo), niobium (Nb) and tantalum (Ta), X is carbon (C), nitrogen (N) or a mixture thereof, n is an integer from 1 to 10, and Tx is one or more selected from a group consisting of oxygen (O), hydroxide (OH), epoxide, C₁₋₅ alkoxide, fluoride (F), chloride (Cl), bromide (Br) and iodide (I) as the terminal functional group of the MXene nanosheet.

Specifically, the MXene nanosheet may be represented by Ti₃C₂Tx (wherein Tx is —OH, —O, —F or a mixture thereof), more specifically by Ti₃C₂Tx (wherein Tx is —OH or —F).

The MXene nanosheet may have an average in-plane diameter of 0.1 to 20 μm and a thickness of 0.5 to 3 nm. Specifically, it may have an average in-plane diameter of 0.7 to 15 μm and a thickness of 0.7 to 2 nm. Most specifically, it may have an average in-plane diameter of 1 to 10 μm and a thickness of 1.2 to 1.8 nm.

If the average in-plane diameter of the MXene nanosheet is smaller than 0.1 μm or if the thickness is smaller than 0.5 nm, electrical conductivity may decline. Conversely, if the average diameter exceeds 20 μm or if the thickness exceeds 3 nm, the quality of the electrode may decline due to decreased thickness uniformity.

The MXene nanosheet is advantageous in that it has large specific surface area and excellent mechanical durability and electrical conductivity and can be processed using an aqueous solution. However, the MXene nanosheet has weak mechanical strength and the electrical conductivity decreases rapidly upon oxidation. Therefore, in the present disclosure, the cellulose nanofiber is bound on the surface of the MXene nanosheet having a layered structure to significantly increase mechanical strength and the surface of the MXene nanosheet is surrounded with the polycarboxylate-based polymer to improve oxidation stability and enhance flexibility at the same time by preventing exposure to oxygen.

The cellulose nanofiber may have an average diameter of 1 to 45 nm and an average length of 0.6 to 15 μm. Specifically, it may have an average diameter of 2 to 40 nm and an average length of 0.8 to 12 μm. Most specifically, it may have an average diameter of 3 to 30 nm and an average length of 1 to 10 μm.

If the cellulose nanofiber has an average diameter smaller than 1 nm or an average length smaller than 0.6 μm, the mechanical strength of the MXene nanosheet may be reduced. Conversely, if the average diameter exceeds 45 nm of if the average length exceeds 15 μm, mechanical strength may be superior, but the MXene nanosheet may be difficult to be applied to a wearable device due to significantly decreased flexibility.

Since the MXene nanosheet has a layered structure, the binding between the layered structure may weaken mechanical strength. Because the cellulose nanofiber has significantly different average diameter and average length from the average diameter and average length of the MXene nanosheet, mechanical loading may be reduced significantly

The polycarboxylate-based polymer may be one or more selected from a group consisting of polycarboxylate ether, polyacrylic acid and polyethylene glycol, specifically polycarboxylate ether, polyacrylic acid or a mixture thereof, most specifically polycarboxylate ether.

The cellulose nanofiber may be hydrogen-bonded to the terminal functional groups present on the surface of the MXene nanosheet, so as to contact the opposing surfaces of the MXene nanosheet. And, the polycarboxylate-based polymer may be covalently bonded to transition metals present on the opposing surfaces of the MXene nanosheet.

In the bioelectrode complex, the MXene nanosheet, the cellulose nanofiber and the polycarboxylate-based polymer may be mixed at a weight ratio of 24:3 to 6:0.5 to 1.8, specifically 24:3 to 5:0.7 to 1.5, most specifically 24:3.8 to 4.2:0.9 to 1.2.

If the content of the cellulose nanofiber is less than 3 in the weight ratio, the mechanical strength of the bioelectrode complex may decline. Conversely, if it exceeds 6 in the weight ratio, the electrical conductivity and flexibility of the bioelectrode complex may decline significantly and there may be limitation in applying the bioelectrode complex to a wearable device due to increased weight.

If the content of the polycarboxylate-based polymer is less than 0.5 in the weight ratio, the bioelectrode complex may be corroded by oxidation due to poor oxidation stability. Conversely, if it exceeds 1.8 in the weight ratio, the bioelectrode complex may not be applied to a wearable device due to declined electrical conductivity and increased weight.

The bioelectrode complex may have an interlayer spacing of 3.3 to 4.2 nm, specifically 3.5 to 4.0 nm, most specifically 3.6 to 3.8 nm, as a result of XRD analysis. If the interlayer spacing is smaller than 3.3 nm, it may be difficult to relieve mechanical impact. Conversely, if it exceeds 4.2 nm, mechanical strength and electrical conductivity may decline.

The bioelectrode complex may have a Ti²⁺/Ti³⁺ area ratio of 3.7 to 4.5, specifically 3.8 to 4.3, most specifically 3.8 to 3.9 nm, as a result of XPS analysis.

Although it was not described explicitly in the examples, comparative examples, etc. given below, after applying bioelectrode complexes for measuring physiological signals according to the present disclosure prepared by varying the following 8 conditions to wearable clothing, deformation and physiological signal sensitivity were evaluated.

As a result, the bioelectrode complex showed very superior deformation and exhibited excellent sensitivity of detecting physiological signals when applied to wearable clothing when all of the following conditions were satisfied.

\textcircled{1} The MXene nanosheet is represented by Ti₃C₂Tx (wherein Tx is —OH, —O or —F). \textcircled{2} The MXene nanosheet has an average in-plane diameter of 1 to 10 μm and a thickness of 1.2 to 1.8 nm. \textcircled{3} The cellulose nanofiber has an average diameter of 3 to 30 nm and an average length of 1 to 10 μm. \textcircled{4} The polycarboxylate-based polymer is polycarboxylate ether. \textcircled{5} The cellulose nanofiber is hydrogen-bonded to the terminal functional groups present on the surface of the MXene nanosheet, so as to contact the opposing surfaces of the MXene nanosheet, and the polycarboxylate-based polymer is covalently bonded to transition metals present on the opposing surfaces of the MXene nanosheet. \textcircled{6} In the bioelectrode complex, the MXene nanosheet, the cellulose nanofiber and the polycarboxylate-based polymer are mixed at a weight ratio of 24:3.8 to 4.2:0.9 to 1.2. \textcircled{7} The bioelectrode complex has an interlayer spacing of 3.6 to 3.8 nm as a result of XRD analysis. \textcircled{8} The bioelectrode complex has a Ti²⁺/Ti³⁺ area ratio of 3.8 to 3.9 as a result of XPS analysis.

If any one of the above 8 conditions was not satisfied, the MXene nanosheet was not suitable to be applied to wearable clothing because of poor deformation and was difficult to be applied to dynamic users because of poor sensitivity of detecting physiological signals.

In addition, the present disclosure provides a wearable device including the bioelectrode complex for measuring physiological signals of the present disclosure.

In addition, the present disclosure provides a clothing for measuring physiological signals, which includes the wearable device of the present disclosure.

In addition, the present disclosure provides a method for manufacturing a bioelectrode complex for measuring physiological signals, which includes: a step of preparing a MXene dispersion containing a MXene nanosheet; a step of preparing a mixture containing the MXene dispersion, a cellulose nanofiber and a polycarboxylate-based polymer; and a step of preparing a bioelectrode complex by coating the mixture on a substrate.

The MXene nanosheet may be represented by Chemical Formula 1.

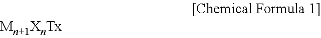

In Chemical Formula 1, M is one or more transition metal selected from a group consisting of titanium (Ti), zirconium (Zr), hafnium (Hf), vanadium (V), chromium (Cr), manganese (Mn), scandium (Sc), molybdenum (Mo), niobium (Nb) and tantalum (Ta), X is carbon (C), nitrogen (N) or a mixture thereof, n is an integer from 1 to 10, and Tx is one or more selected from a group consisting of oxygen (O), hydroxide (OH), epoxide, C₁₋₅ alkoxide, fluoride (F), chloride (Cl), bromide (Br) and iodide (I) as the terminal functional group of the MXene nanosheet.

Specifically, the MXene nanosheet may be represented by Ti₃C₂Tx (wherein Tx is —OH, —O, —F or a mixture thereof), more specifically by Ti₃C₂Tx (wherein Tx is —OH or —F).

The MXene nanosheet may have an average in-plane diameter of 0.1 to 20 μm and a thickness of 0.5 to 3 nm. Specifically, it may have an average in-plane diameter of 0.7 to 15 μm and a thickness of 0.7 to 2 nm. Most specifically, it may have an average in-plane diameter of 1 to 10 μm and a thickness of 1.2 to 1.8 nm.

The cellulose nanofiber may have an average diameter of 1 to 45 nm and an average length of 0.6 to 15 μm. Specifically, it may have an average diameter of 2 to 40 nm and an average length of 0.8 to 12 μm. Most specifically, it may have an average diameter of 3 to 30 nm and an average length of 1 to 10 μm.

The polycarboxylate-based polymer may be one or more selected from a group consisting of polycarboxylate ether, polyacrylic acid and polyethylene glycol, specifically polycarboxylate ether, polyethylene glycol or a mixture thereof, most specifically polycarboxylate ether.

The cellulose nanofiber is hydrogen-bonded to the terminal functional groups of the MXene nanosheet, such as hydroxyl groups, oxygen groups, fluorine groups, etc., so as to contact the opposing surfaces of the MXene nanosheet. And, the polycarboxylate-based polymer is covalently bonded to transition metals present on the opposing surfaces of the MXene nanosheet.

In the bioelectrode complex, the MXene nanosheet, the cellulose nanofiber and the polycarboxylate-based polymer may be mixed at a weight ratio of 24:3 to 6:0.5 to 1.8, specifically 24:3 to 5:0.7 to 1.5, most specifically 24:3.8 to 4.2:0.9 to 1.2.

The bioelectrode complex may have an interlayer spacing of 3.3 to 4.2 nm, specifically 3.5 to 4.0 nm, most specifically 3.6 to 3.8 nm, as a result of XRD analysis.

The bioelectrode complex may have a Ti²⁺/Ti³⁺ area ratio of 3.7 to 4.5, specifically 3.8 to 4.3, most specifically 3.8 to 3.9, as a result of XPS analysis.

Hereinafter, the present disclosure will be described more specifically through examples. However, the present disclosure is not limited by the examples.

### Example 1 and Comparative Examples 1 to 3: Preparation of MX-CNF-PCE Electrode Complex

An etchant solution was prepared in a 250-mL Teflon beaker as follows. 3.2 g of LiF (99.9%; Sigma-Aldrich) was dissolved in 40 mL of a 9 M HCl aqueous solution and cooled to <5° C. using an ice-water bath. The etchant solution was then deoxygenated with ultrahigh-purity N2. Subsequently, 2.0 g of MAX phase (Ti₃AIC2 powder, 99%, 325 mesh; Forsman) was gradually added to the etchant solution under gentle stirring. The mixture was deoxygenated again and protected in an inert atmosphere. Etching was performed in an oil bath at 25° C. for 48 hours to remove most of the Al layer from the MAX phase. The resulting product was washed and delaminated in two 50 mL centrifuge tubes using deoxygenated water and then subjected to five to six cycles of centrifugation and shaking. Finally, the delaminated MXene was sonicated in an ice bath for 30 minutes and centrifuged at 2826×g for 30 minutes to obtain a supernatant predominantly composed of single-layer MXene. The supernatant was a Ti₃C₂Tx MXene (wherein Tx is —OH, —O or F) dispersion. The Ti₃C₂Tₓ MXene had an average in-plane diameter of 1 to 10 μm and a thickness of 1.2 to 1.8 nm.

The MXene dispersion was mixed with a CNF (cellulose nanofiber) polymer aqueous solution (1 wt %; average diameter=3 to 30 nm, average length=1 to 10 μm) and a PCE (polycarboxylate ether) polymer aqueous solution (Mw=550,000 g/mol, 55.0 wt %) according to the weight ratio specified in the experimental design (MXene:CNF:PCE=24:4:1 (Example 1), 24:2:1 (Comparative Example 1), 24:4:2 (Comparative Example 2), and 24:2:2 (Comparative Example 3)). The mixture of MXene and polymers was vigorously shaken for 30 minutes to obtain a homogeneous MX-CNF-PCE electrode complex solution. An MX-CNF-PCE electrode complex solution was prepared by coating the MX-CNF-PCE electrode complex solution on a substrate.

### Test Example 1: SEM and EDX Elemental Mapping Analyses of MX-CNF-PCE Electrode Complex

The surface morphology and elemental distribution of the MX-CNF-PCE electrode complex prepared in Example 1 were investigated by SEM (JSM-7610F-Plus) and EDX analyses. The result is shown in FIGS. 2, 3 and 4A to 4E.

FIG. 2 shows the SEM and EDX elemental mapping results of the MX-CNF-PCE electrode complex prepared in Example 1.

FIG. 3 shows the SEM image of a pristine cotton fabric.

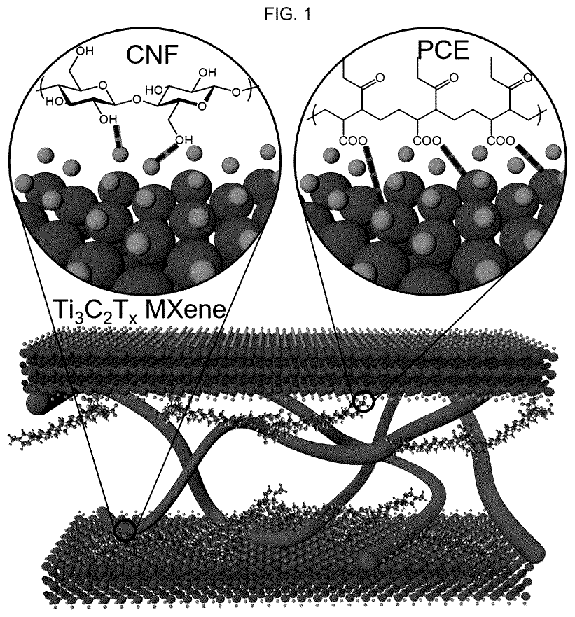

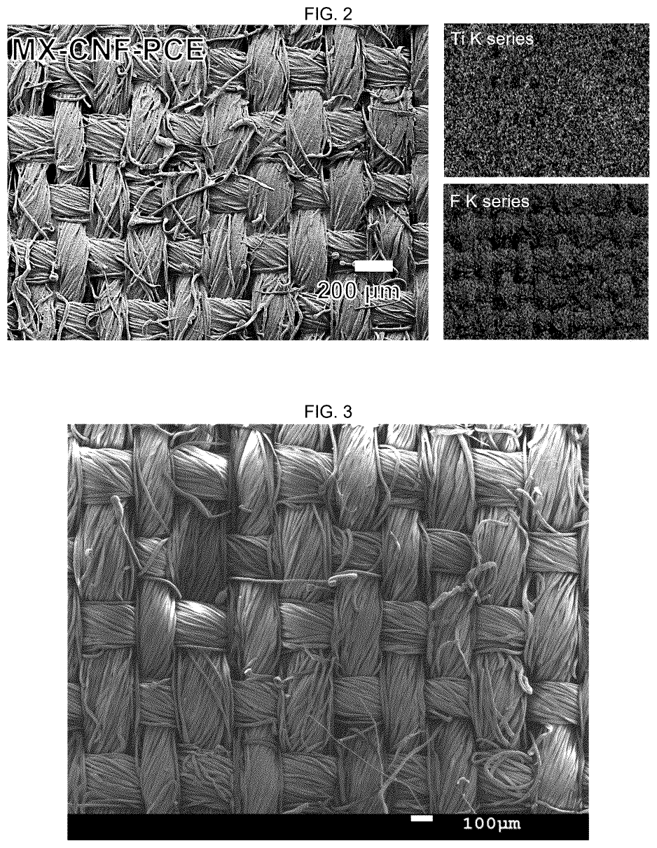

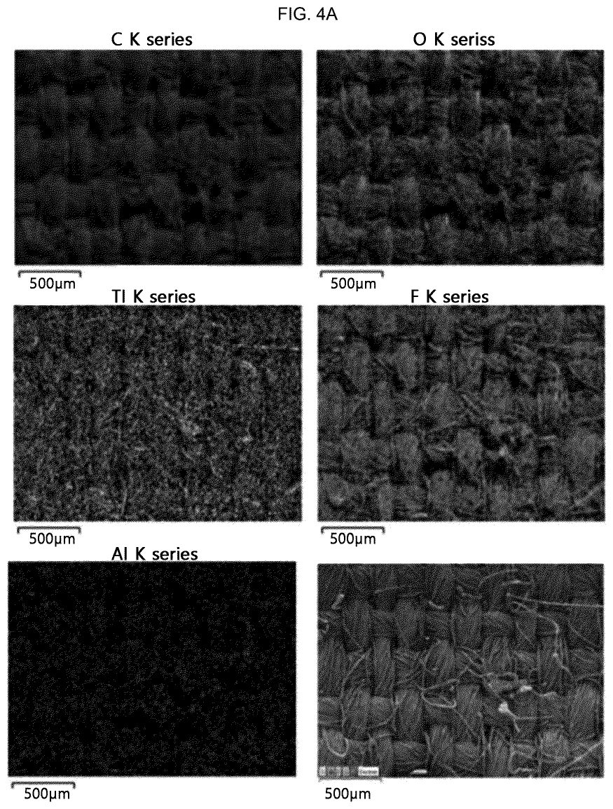

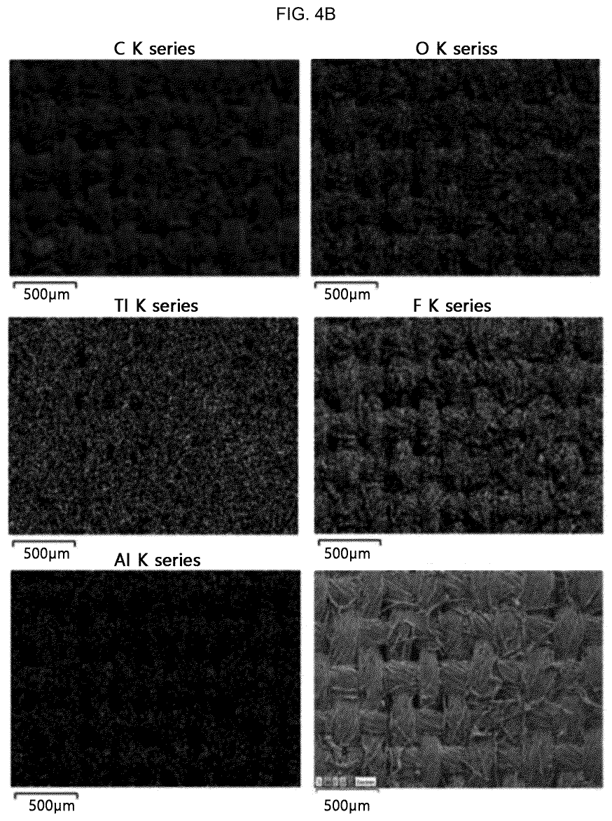

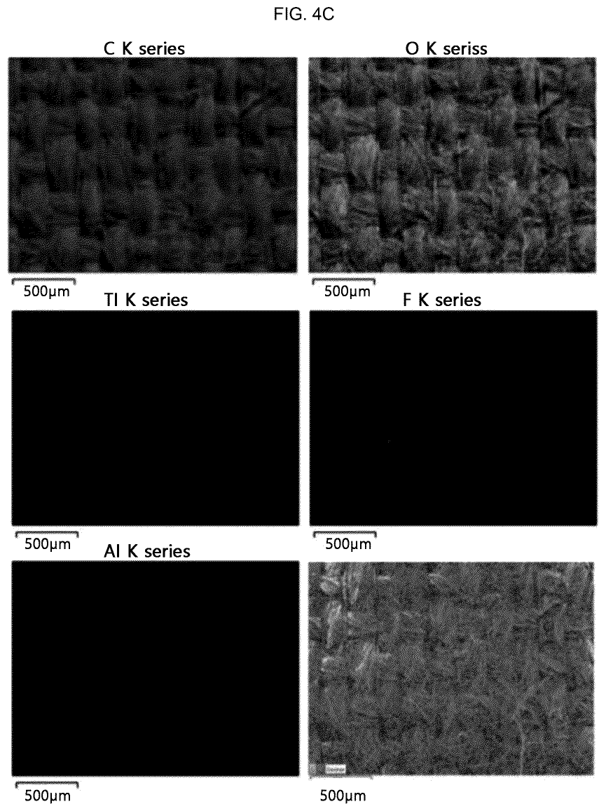

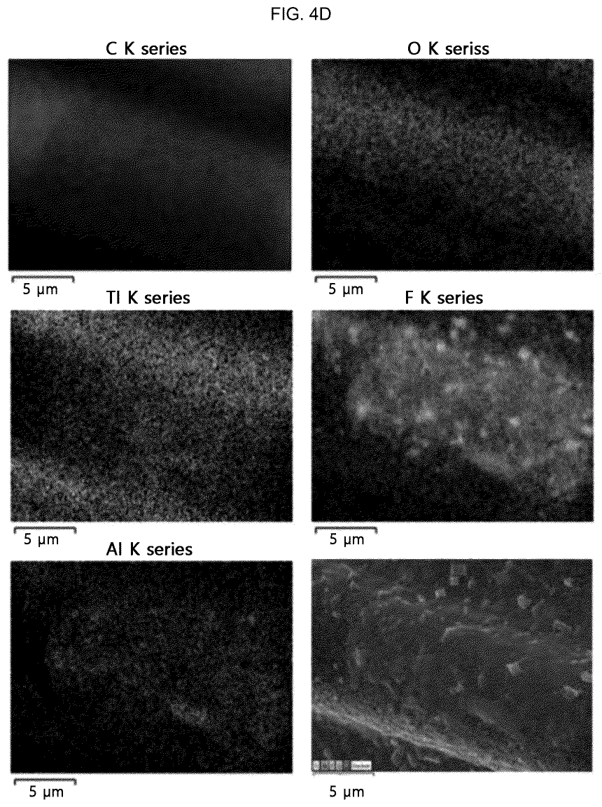

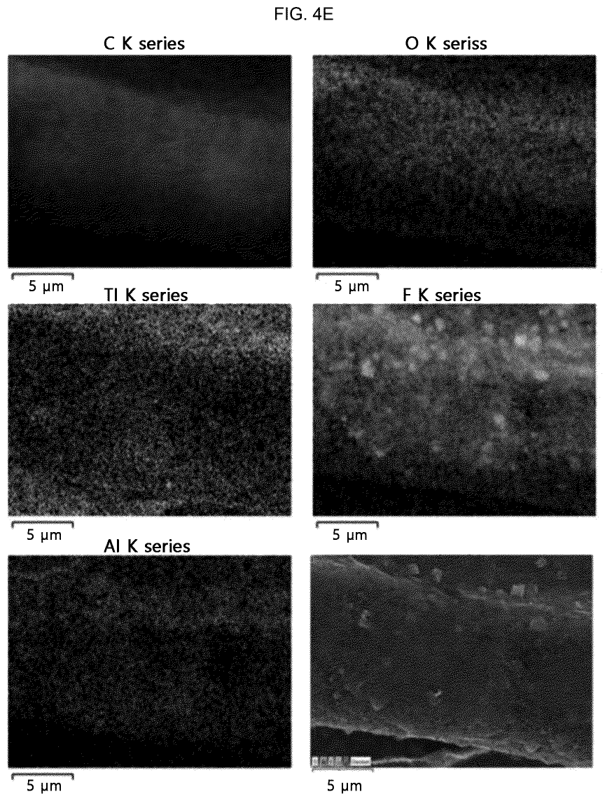

FIG. 4A shows the EDX mapping result of pristine MXene electrodes at 50× magnification.

FIG. 4B shows the EDX mapping result of MX-CNF-PCE electrode complexes at 50× magnification.

FIG. 4C shows the EDX mapping result of cotton fabrics at 50× magnification.

FIG. 4D shows the EDX mapping result of cotton fabrics at 5000× magnification.

FIG. 4E shows the EDX mapping result of MX-CNF-PCE electrode complexes at 5000× magnification.

Referring to FIGS. 2, 3 and 4A to 4E, as a result of investigating the uniformity and integrity of the spray-coated MX-CNF-PCE electrode complex solution by scanning electron microscopy (SEM) and energy-dispersive X-ray (EDX) analysis, it was confirmed that the MXene nanosheets enveloped the cotton fibers and bridged the gaps between them, while the pristine cotton fabric of FIG. 3 exhibited a clean surface characterized by interwoven warp and weft yarns. Additionally, the uniform distribution of each element in the MXene-based solution was observed from the EDX mapping result shown in FIGS. 4A to 4E.

### Test Example 2: Analysis of Resistance of MX-CNF-PCE Electrode Complex Between Skin and Electrode

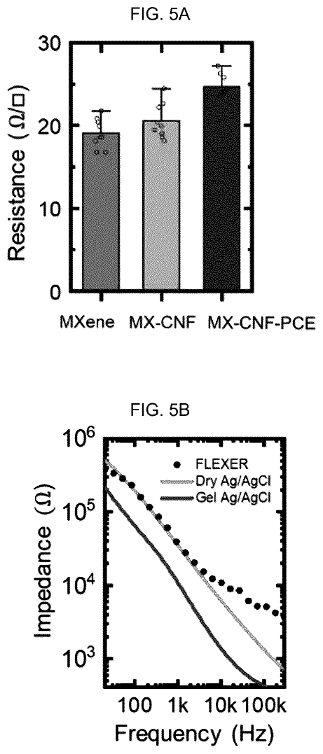

The sheet resistance between the skin and the electrode was measured using the four-point probe method (Keithley 2182A, 2634B and 6221) by applying the MX-CNF-PCE electrode complex prepared in Example 1 to wearable FLEXER. The skin-to-electrode impedance was measured using an inductance-capacitance-resistance (LCR) meter (E-4980AL; KEYSIGHT) in the frequency range of 20 Hz to 300 kHz. The result is shown in FIGS. 5A and 5B.

FIG. 5A shows the result of comparing the sheet resistance of MXene-based electrodes, i.e., a MXene electrode, an MX-CNF electrode and an MX-CNF-PCE electrode complex. The bar chart shows sample means (n=8) with standard deviation error bars.

Generally, electrodes must have low sheet resistance to be able to measure physiological signals with high reliability. Referring to FIG. 5A, the MX-CNF-PCE electrode complex and the MX-CNF electrode showed sheet resistances of 24.7Ω and 20.5Ω, respectively, which were slightly higher than that of the MXene electrode (19.1Ω). This is attributed to the inclusion of electrically nonconductive CNF and PCE within the MXene interlayers. However, the sheet resistances are still significantly lower than those of the existing MXene-coated cotton electrodes (569 to 761Ω).

FIG. 5B shows the result of comparing skin impedance between reference dry and wet Ag/AgCl electrodes and pneumatically fully activated FLEXER for the MXene electrode, the MX-CNF electrode and the MX-CNF-PCE electrode complex.

In addition to the sheet resistance of electrodes, the skin-to-electrode impedance is also a crucial factor. The skin-to-electrode impedance reflects the skin's resistance in transmitting electrical signals to the sensing element of the electrode. A low skin-to-electrode impedance is associated with high signal quality, as it signifies reduced resistance at the contact point between the skin and conductive surface. FIG. 5B shows the measured skin-to-electrode contact impedances of the FLEXER electrodes using the MX-CNF-PCE electrode complex and reference dry and gel-type Ag/AgCl electrodes.

The inflated FLEXER electrode displayed skin-to-electrode impedance comparable to those of the reference electrodes of the same size. Notably, the skin-to-electrode impedance of the FLEXER electrode was similar to that of the dry electrode, up to a frequency of 2.45 kHz. At a frequency of 100 Hz, the FLEXER electrode exhibited an impedance of 191 kΩ, while the dry and gel-type Ag/AgCl electrodes exhibited impedances of 190 kΩ and 64 kΩ, respectively. These impedance values are sufficiently low for accurate signal measurements without significant noise interference.

### Test Example 3: Evaluation of Tensile Strain and Bending Stability of MX-CNF-PCE Electrode

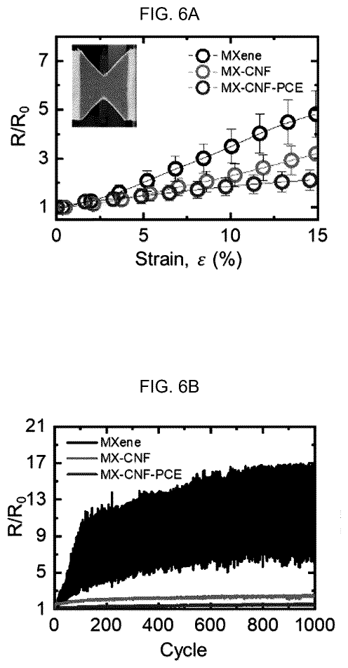

The tensile strain and bending stability of the MX-CNF-PCE electrode complexes were measured using a stepper motor controller (ECOPIA, BM-111), and the result is shown in FIGS. 6A and 6B.

FIG. 6A shows the tensile strain of the MXene electrode, the MX-CNF electrode and the MX-CNF-PCE electrode complex. Sample means (n=5) are shown with standard deviation error bars.

The electromechanical response of the MX-CNF-PCE electrode complex was investigated under mechanical deformation. A 15% tensile strain was applied to the three electrodes (MXene electrode, MX-CNF electrode and MX-CNF-PCE electrode complex), and the normalized resistance (R/Ro) was measured as shown in FIG. 6A. The MX-CNF-PCE electrode complex showed the smallest R/Ro increase of 2.1 at a 15% tensile strain, whereas the MXene electrode and the MX-CNF electrode exhibited R/Ro values of 4.8 and 3.2, respectively. The enhanced stability of the MX-CNF-PCE electrode complex is attributed to its unique brick-and-mortar structure. In this structure, the 2D MXene nanosheets function as bricks, while the 1D CNF serves as the mortar. The incorporation of the synthetic polymer PCE significantly enhanced mechanical stability through additional hydrogen bonds. Owing to these improvements, the MX-CNF-PCE electrode complex exhibits a decrease in R/Ro in contrast to the MX-CNF electrode.

FIG. 6B shows the result of 1000 cycles of 15% bending strain test for the MXene electrode, the MX-CNF electrode and the MX-CNF-PCE electrode complex.

FIG. 6B shows the result of conducting a cyclic bending stability test by subjecting the three MXene-based electrodes to a 15% strain repeatedly for 1000 cycles. This test simulates situations where stress is frequently applied to specific areas, such as knees or elbows. A consistent trend was observed throughout the test. The inclusion of CNF and PCE in the MX-CNF-PCE electrode complex enhanced electromechanical stability, with the R/Ro values of the MX-CNF-PCE electrode complex, the MX-CNF electrode and the MXene electrode being 1.4 to 1.6, 2.3 to 2.6, and 6.1 to 16.9, respectively.

### Test Example 4: Evaluation of Electromechanical Stability of MX-CNF-PCE Electrode Complex

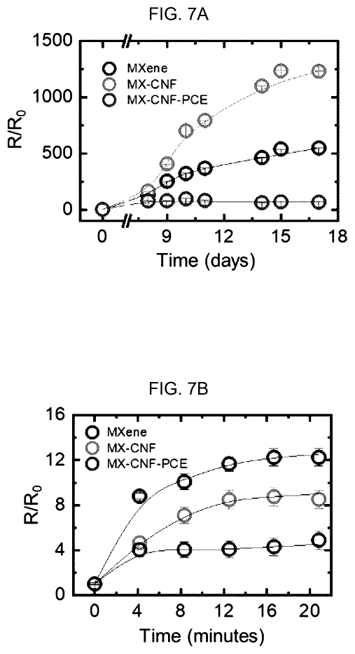

In order to confirm the electromechanical stability of the MX-CNF-PCE electrode complex, oxidation stability under 100% humidity environment and resistance stability against artificial sweat droplets were evaluated. All electromechanical resistance data of the MXene-based electrodes were gathered using a parameter analyzer (4200A-SCS; Keithley). The result is shown in FIG. 7.

FIG. 7A shows the result of evaluating the oxidation stability of the MXene electrode, the MX-CNF electrode and the MX-CNF-PCE electrode complex under 100% humidity environment. Sample means (n=5) are shown with standard deviation error bars.

For the oxidation resistance test, the MXene-based electrodes were exposed to a sealed glass chamber with 100% relative humidity for 17 days, as shown in FIG. 7A. The MX-CNF-PCE electrode complex showed remarkable oxidation stability, with the R/Ro value increased modestly from 1 to 65. The increase in the R/Ro value was significant for the MXene electrode and the MX-CNF electrode, from 1 to 1231 and 548 (20 times and 9 times that of the MX-CNF-PCE electrode complex), respectively. This is attributed to the protective barrier formed by the incorporation of PCE, which effectively shields the MXene electrode from oxidation. It can be seen that sweat excreted by the skin can also affect the performance of the bioelectrodes, thereby affecting their R/Ro ratio.

FIG. 7B shows the resistance stability of the MXene electrode, the MX-CNF electrode and the MX-CNF-PCE electrode complex to artificial sweat droplets. Sample means (n=5) are shown with standard deviation error bars.

To assess the stability of the MX-CNF-PCE electrode complex in response to perspiration, its electrical characteristics were observed in artificial sweat, as shown in FIG. 7B. After wetting the three MXene-based electrodes with artificial sweat, their electrical response (R/Ro) was monitored for 20 minutes. As a result, the MX-CNF-PCE electrode complex exhibited the most electrically stable response because of the PCE coating on the MXene nanosheets.

### Test Example 5: Evaluation of Mechanical Properties and Stability of MX-CNF-PCE Electrode Complex Depending on Mixing Ratio of CNF and PCE

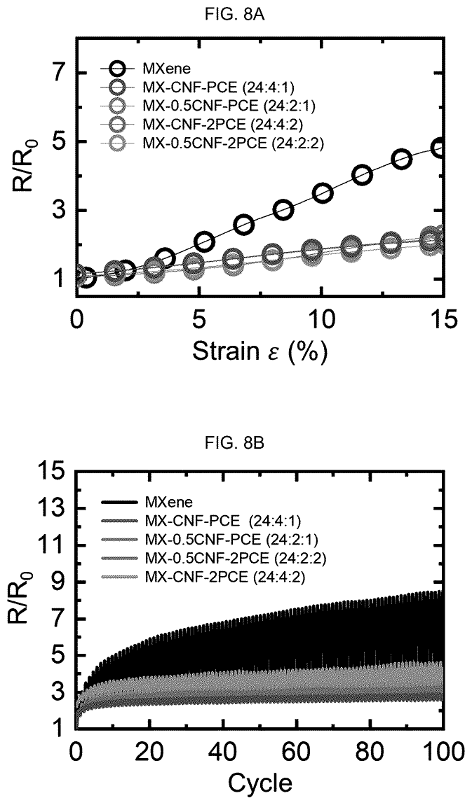

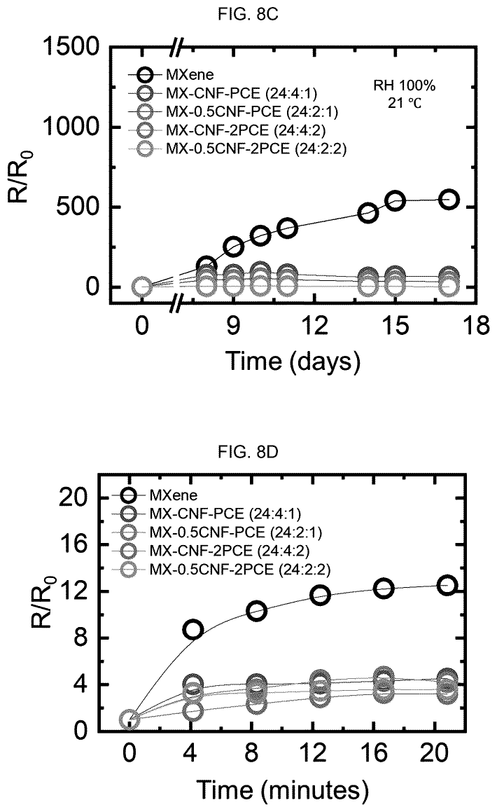

Experiments were conducted to determine the optimal mixing ratio of the MX-CNF-PCE electrode complex for enhanced electromechanical properties by controlling the amounts of CNF and PCE. Mechanical properties and stability were evaluated for the MX-CNF-PCE electrode complexes prepared in Example 1 and Comparative Examples 1 to 3 by mixing MX, CNF and PCE at a weight ratio of 24:4:1, 24:2:1, 24:4:2, and 24:2:2, respectively, according to the methods described in Test Examples 3 and 4, and the result is shown in FIGS. 8A to 8D.

FIG. 8A shows the tensile strain of the MXene electrode and the MX-CNF-PCE electrode complexes prepared in Example 1 and Comparative Examples 1 to 3 depending on the mixing ratio of CNF and PCE.

FIG. 8B shows the result of cyclic bending for the MXene electrode and the MX-CNF-PCE electrode complexes prepared in Example 1 and Comparative Examples 1 to 3.

FIG. 8C shows the oxidation stability of the MXene electrode and the MX-CNF-PCE electrode complexes prepared in Example 1 and Comparative Examples 1 to 3 under 100% humidity environment.

FIG. 8D shows the resistance stability of the MXene electrode and the MX-CNF-PCE electrode complexes prepared in Example 1 and Comparative Examples 1 to 3 against artificial sweat droplets.

Since CNF and PCE act as insulators for the electrode in the MX-CNF-PCE electrode complex, their proportion needs to be minimized in comparison to the MXene electrode. Considering the spray coating capability and electrical conductivity of the bioelectrode, the optimized weight ratio of the MXene electrode was determined to 82 wt % of the total electrode composition. Maintaining this MXene electrode weight ratio (82 wt %), the amounts of CNF and PCE in the electrode was adjusted precisely to further improve its environmental stability. By comparing the environmental stability of the bioelectrodes with different CNF and PCE ratios, the weight ratio of 24:4:1 (MXene:CNF:PCE) was identified as optimal for the FLEXER system. Consequently, it was confirmed that the MX-CNF-PCE electrode complex has enhanced electromechanical and environmental resistance properties under various mechanical deformations, including tensile strain and cyclic bending, 100% humidity environment, and interference from sweat.

### Test Example 6: Analysis of XRD and XPS of MX-CNF-PCE Electrode Complex

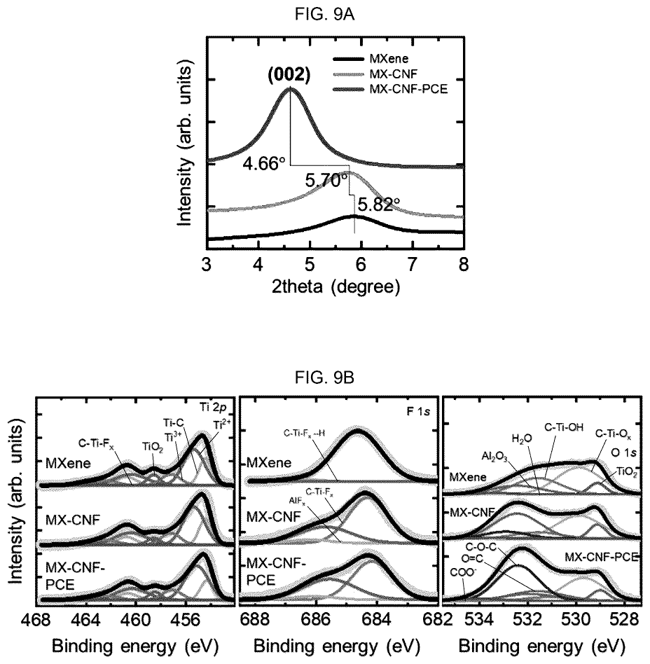

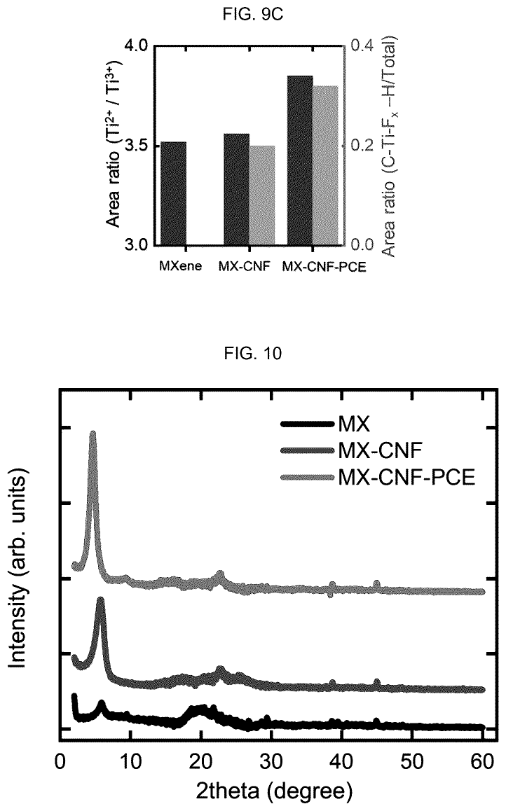

XRD and XPS analyses were conducted to investigate the interlayer spacing and chemical structure of the MX-CNF-PCE electrode complex, and the result is shown in FIGS. 9A to 9C and 10. XRD (SmartLab; Rigaku) was used to detect the differences in the interlayer spacing of the MXene-based electrodes. An X-ray photoelectron spectrometer (K-alpha, Thermo U.K.) was operated using an aluminum anode as a source with a spot size of 400 μm.

FIG. 9A shows the XRD spectra of the MXene electrode, the MX-CNF electrode and the MX-CNF-PCE electrode complex.

FIG. 9B shows the XPS spectra of the MXene electrode, the MX-CNF electrode and the MX-CNF-PCE electrode complex.

FIG. 9C compares the XPS analysis result of Ti²⁺/Ti³⁺ area ratio and hydrogen bond ratio for the MXene electrode, the MX-CNF electrode and the MX-CNF-PCE electrode complex referred from the area of XPS deconvoluted peaks.

FIG. 10 shows the extended XRD spectra of the MXene electrode, the MX-CNF electrode and the MX-CNF-PCE electrode complex.

Referring to FIG. 9A and FIG. 10, the X-ray diffraction (XRD) curves reveal the evidence of the intercalation and blending of CNF and PCE within the MXene nanosheets. Specifically, the (002) diffraction peak of MXene appeared at 26=₅0.82°, which shifted to 5.70° in MX-CNF and 4.66° in MX-CNF-PCE. These shifts correspond to increase in the interlayer spacing from 3.04 nm to 3.10 nm and 3.78 nm, respectively.

FIG. 9B shows the X-ray photoelectron spectroscopy (XPS) spectra of the MXene electrode, the MX-CNF electrode and the MX-CNF-PCE electrode complex. As a result of the analysis of the Ti 2p and O 1s spectra, it was revealed that the area ratio of Ti²⁺ to Ti³⁺ peaks increased from 3.52 in the MXene electrode to 3.56 and 3.85 in the MX-CNF electrode and the MX-CNF-PCE electrode complex, respectively. Simultaneously, the area ratio of the —COO— peak to the total chemical bonds decreased from 0.04 in the MXene electrode to 0.02 in the MX-CNF electrode and the MX-CNF-PCE electrode complex. These observations confirm the formation of covalent Ti—O bonds between the MXene nanosheets and the PAA anchor of PCE, as revealed by the Ti 2p spectrum (i.e., —COOH). Furthermore, the F 1s spectrum reveals the presence of hydrogen bonds between the MXene nanosheets and the hydroxyl groups in CNF or the partially hydrolyzed —COO— of PCE (i.e., —COOH).

The area ratio of the hydrogen bond peak (C—Ti-Fx-H) to the total area increased from 0 in the MXene electrode to 0.2 and 0.32 in the MX-CNF electrode and the MX-CNF-PCE electrode complex, respectively. The appearance of a high binding energy peak in the F is region for C—Ti-Fx-H demonstrates the formation of hydrogen bonds, which contributed to the MX-CNF electrode and the MX-CNF-PCE electrode complex exhibiting better mechanical stability than the MXene electrode. These ratio increments are shown in FIG. 9C.

What is claimed is:

1. A bioelectrode complex for measuring physiological signals, comprising:
a MXene nanosheet having a layered structure; and
a cellulose nanofiber and a polycarboxylate-based polymer located between the MXene nanosheet and bound on the surface of the MXene nanosheet.

2. The bioelectrode complex for measuring physiological signals according to claim 1, wherein the MXene nanosheet is represented by Chemical Formula 1: 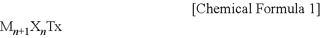
wherein
M is one or more transition metal selected from a group consisting of titanium (Ti), zirconium (Zr), hafnium (Hf), vanadium (V), chromium (Cr), manganese (Mn), scandium (Sc), molybdenum (Mo), niobium (Nb) and tantalum (Ta),
X is carbon (C), nitrogen (N) or a mixture thereof,
n is an integer from 1 to 10, and
Tx is one or more selected from a group consisting of oxygen (O), hydroxide (OH), epoxide, C₁₋₅ alkoxide, fluoride (F), chloride (Cl), bromide (Br) and iodide (I) as the terminal functional group of the MXene nanosheet.

3. The bioelectrode complex for measuring physiological signals according to claim 1, wherein the MXene nanosheet has an average in-plane diameter of 0.1 to 20 μm and a thickness of 0.5 to 3 nm.

4. The bioelectrode complex for measuring physiological signals according to claim 1, wherein the cellulose nanofiber has an average diameter of 1 to 45 nm and an average length of 0.6 to 15 μm.

5. The bioelectrode complex for measuring physiological signals according to claim 1, wherein the polycarboxylate-based polymer is one or more selected from a group consisting of polycarboxylate ether, polyacrylic acid and polyethylene glycol.

6. The bioelectrode complex for measuring physiological signals according to claim 1, wherein the cellulose nanofiber is hydrogen-bonded to the terminal functional groups present on the surface of the MXene nanosheet, so as to contact the opposing surfaces of the MXene nanosheet, and the polycarboxylate-based polymer is covalently bonded to transition metals present on the opposing surfaces of the MXene nanosheet.

7. The bioelectrode complex for measuring physiological signals according to claim 1, wherein, in the bioelectrode complex, the MXene nanosheet, the cellulose nanofiber and the polycarboxylate-based polymer are mixed at a weight ratio of 24:3 to 6:0.5 to 1.8.

8. The bioelectrode complex for measuring physiological signals according to claim 1, wherein the bioelectrode complex has an interlayer spacing of 3.3 to 4.2 nm as a result of XRD analysis.

9. The bioelectrode complex for measuring physiological signals according to claim 1, wherein the bioelectrode complex has a Ti²⁺/Ti³⁺ area ratio of 3.7 to 4.5 as a result of XPS analysis.

10. The bioelectrode complex for measuring physiological signals according to claim 1, wherein
the MXene nanosheet is represented by Ti₃C₂Tx (wherein Tx is —OH, —O or —F),
the MXene nanosheet has an average in-plane diameter of 1 to 10 μm and a thickness of 1.2 to 1.8 nm,
the cellulose nanofiber has an average diameter of 3 to 30 nm and an average length of 1 to 10 μm,
the polycarboxylate-based polymer is polycarboxylate ether,
the cellulose nanofiber is hydrogen-bonded to the terminal functional groups present on the surface of the MXene nanosheet, so as to contact the opposing surfaces of the MXene nanosheet, and the polycarboxylate-based polymer is covalently bonded to transition metals present on the opposing surfaces of the MXene nanosheet,
in the bioelectrode complex, the MXene nanosheet, the cellulose nanofiber and the polycarboxylate-based polymer are mixed at a weight ratio of 24:3.8 to 4.2:0.9 to 1.2,
the bioelectrode complex has an interlayer spacing of 3.6 to 3.8 nm as a result of XRD analysis, and
the bioelectrode complex has a Ti²⁺/Ti³⁺ area ratio of 3.8 to 3.9 as a result of XPS analysis.

11. A wearable device comprising the bioelectrode complex for measuring physiological signals according to claim 1.

12. A clothing for measuring physiological signals comprising the wearable device according to claim 11.

13. A method for manufacturing a bioelectrode complex for measuring physiological signals, comprising:
a step of preparing a MXene dispersion containing a MXene nanosheet;
a step of preparing a mixture containing the MXene dispersion, a cellulose nanofiber and a polycarboxylate-based polymer; and
a step of preparing a bioelectrode complex by coating the mixture on a substrate.

14. The method for manufacturing a bioelectrode complex for measuring physiological signals according to claim 13, wherein the MXene nanosheet is represented by Chemical Formula 1: 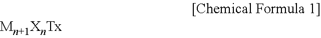
wherein
M is one or more transition metal selected from a group consisting of titanium (Ti), zirconium (Zr), hafnium (Hf), vanadium (V), chromium (Cr), manganese (Mn), scandium (Sc), molybdenum (Mo), niobium (Nb) and tantalum (Ta),
X is carbon (C), nitrogen (N) or a mixture thereof,
n is an integer from 1 to 10, and
Tx is one or more selected from a group consisting of oxygen (O), hydroxide (OH), epoxide, C₁₋₅ alkoxide, fluoride (F), chloride (Cl), bromide (Br) and iodide (I) as the terminal functional group of the MXene nanosheet.

15. The method for manufacturing a bioelectrode complex for measuring physiological signals according to claim 13, wherein the cellulose nanofiber has an average diameter of 1 to 45 nm and an average length 0.6 to 15 μm.

16. The method for manufacturing a bioelectrode complex for measuring physiological signals according to claim 13, wherein the polycarboxylate-based polymer is one or more selected from a group consisting of polycarboxylate ether, polyacrylic acid and polyethylene glycol.

17. The method for manufacturing a bioelectrode complex for measuring physiological signals according to claim 13, wherein the cellulose nanofiber is hydrogen-bonded to the terminal functional groups present on the surface of the MXene nanosheet, so as to contact the opposing surfaces of the MXene nanosheet, and the polycarboxylate-based polymer is covalently bonded to transition metals present on the opposing surfaces of the MXene nanosheet.

18. The method for manufacturing a bioelectrode complex for measuring physiological signals according to claim 13, wherein, in the bioelectrode complex, the MXene nanosheet, the cellulose nanofiber and the polycarboxylate-based polymer are mixed at a weight ratio of 24:3 to 6:0.5 to 1.8.

19. The method for manufacturing a bioelectrode complex for measuring physiological signals according to claim 13, wherein the bioelectrode complex has an interlayer spacing of 3.3 to 4.2 nm as a result of XRD analysis.

20. The method for manufacturing a bioelectrode complex for measuring physiological signals according to claim 13, wherein
the MXene nanosheet is represented by Ti₃C₂Tx (wherein Tx is —OH, —O or —F),
the MXene nanosheet has an average in-plane diameter of 1 to 10 μm and a thickness of 1.2 to 1.8 nm,
the cellulose nanofiber has an average diameter of 3 to 30 nm and an average length of 1 to 10 μm,
the polycarboxylate-based polymer is polycarboxylate ether,
the cellulose nanofiber is hydrogen-bonded to the terminal functional groups present on the surface of the MXene nanosheet, so as to contact the opposing surfaces of the MXene nanosheet, and the polycarboxylate-based polymer is covalently bonded to transition metals present on the opposing surfaces of the MXene nanosheet,
in the bioelectrode complex, the MXene nanosheet, the cellulose nanofiber and the polycarboxylate-based polymer are mixed at a weight ratio of 24:3 to 6:0.5 to 1.8,
the bioelectrode complex has an interlayer spacing of 3.6 to 3.8 nm as a result of XRD analysis, and
the bioelectrode complex has a Ti²⁺/Ti³⁺ area ratio of 3.8 to 3.9 as a result of XPS analysis.
